(19) United States
(12) Patent Application Publication
(10) Pub. No.: US 2026/0227359 A1
(43) Pub. Date: Aug. 6, 2026

(54) METHOD FOR MONITORING FIBERS INSIDE CONCRETES, SYSTEM, MEDIUM AND PRODUCT

(71) Applicant: Qingdao University of Technology, Shandong (CN)

(72) Inventors: Peng Zhang, Shandong (CN); Zihao Yu, Shandong (CN); Ling Qin, Shandong (CN); Wentao Wang, Shandong (CN); Jiuwen Bao, Shandong (CN)

(73) Assignee: Qingdao University of Technology, Shandong (CN)

(21) Appl. No.: 18/861,523

(22) PCT Filed: Mar. 18, 2024
(86) PCT No.: PCT/CN2024/093239

§ 371 Date: Oct. 29, 2024

(30) Foreign Application Priority Data
Mar. 18, 2024 (CN) 202410304564.8

(51) Int. Cl. G01N 27/22; G01N 33/38

(52) U.S. Cl. CPC G01N 27/221; G01N 27/226; G01N 33/383

(57) ABSTRACT

A method for monitoring fibers inside concretes, a system, a medium and a product are provided, relating to the technical field of concrete material monitoring. The method includes: acquiring a concrete to be measured; measuring a capacitance value of the concrete to be measured by using a capacitive sensor; inputting the capacitance value of the concrete to be measured into a permittivity calculation formula to obtain a permittivity of the concrete to be measured; acquiring a permittivity of a target concrete and a permittivity of a fiber-free concrete; and inputting the permittivity of the concrete to be measured, the permittivity of the target concrete and the permittivity of the fiber-free concrete into a prediction model, so as to obtain a measured fiber direction effective coefficient of the concrete to be measured as a monitoring result of the concrete to be measured. The method can monitor fibers inside concretes accurately, rapidly and quantitatively.

### CROSS-REFERENCE TO RELATED APPLICATION

This patent application is a national stage application of International Patent Application No. PCT/CN2024/093239, filed on May 15, 2024, which claims the benefit and priority of Chinese Patent Application No. 202410304564.8 filed with the China National Intellectual Property Administration on Mar. 18, 2024, and entitled as “METHOD FOR MONITORING FIBERS INSIDE CONCRETES, SYSTEM, MEDIUM AND PRODUCT”, the disclosure of which is incorporated by reference herein in its entirety as part of the present application.

### BACKGROUND OF THE INVENTION

### Field of the Invention

One or more embodiments of the invention relate to the technical field of concrete material monitoring, in particular to a method for monitoring fibers inside concretes, a system, a medium and a product.

### Description of the Related Art

Fiber concretes have been widely used in modern civil engineering construction due to its remarkable characteristics such as enhanced toughness, delayed crack propagation and improved seismic resistance. However, the distribution uniformity, the direction and the density of fibers inside concretes have a decisive influence on its performance. The non-uniform fiber distribution may lead to the material properties lower than expected, and even lead to an initial structural damage. Therefore, in order to ensure the expected performance of concretes and provide reliable guarantee for long-term service, it is particularly critical to monitor and analyze the fiber arrangement inside concretes accurately, quickly and in real time.

A capacitive sensor senses the existence, position and other properties of objects based on the change of capacitance. The signal change of the capacitive sensor depends on the distance between electrodes, the area of electrodes and the medium of a measurement field. Therefore, commonly used capacitive sensors are divided into variable distance-type sensors, variable area-type sensors and variable permittivity-type sensors. A capacitive sensor has been widely used in the aspects such as the industrial field, the medical field and the meteorological monitoring field because of its advantages of non-contact, high sensitivity, fast response, strong applicability and versatility.

At present, it is a common technology to evaluate the fiber arrangement effect by measuring the change of the internal resistance of concretes using a resistance method. However, the resistance method has some limitations, such as sensitivity to humidity and temperature, which may affect the measurement accuracy. In addition, the resistance method usually needs to be in contact with the measured part, which cannot realize nondestructive monitoring. In contrast, a capacitance method shows obvious advantages in detecting fibers. First, the capacitance method is non-invasive and can be measured without touching or damaging the concrete structure, which is very important for protecting the integrity of the structure. Second, the capacitance method is less sensitive to environmental factors such as humidity and temperature, so that the capacitance method can provide more stable and reliable measurement results. In addition, the capacitance method can provide faster response time, which is especially important for real-time monitoring. Therefore, a fiber distribution monitoring technology based on a capacitance method is developed, which can overcome the limitations of the resistance method and provide more accurate and efficient monitoring means.

In the capacitance method, some properties or states of the medium can be inferred by measuring the capacitance value of the medium. The capacitance method is suitable for many fields, such as metallurgy, electric power, chemical industry, medicine and energy, and is used to monitor the characteristics, the mixing process and the diffusion process of materials. In the application of fiber concrete materials, the capacitance method can detect the distribution and arrangement of internal fibers by measuring the change of the capacitance value within concretes. The non-invasive and quick response characteristics of the technology enable it to monitor the internal changes of concretes in real time, thus providing important information in the fields of architecture and engineering. The advantages include non-invasive characteristics, a quick response, a wide application range, a low cost and without no need to worry about radiation problems. Because of these advantages, the capacitance method shows great potential in detecting the internal fiber arrangement of fiber concretes. In particular, the non-invasive and fast response characteristics make it have obvious advantages in real-time monitoring and detecting distribution of fibers inside concretes.

In recent years, with the continuous progress of technology, the capacitance method has been widely used in many fields, but its application in the research of fiber concretes is still relatively limited. Therefore, developing a method that can provide quantitative data for fiber distribution in fiber concretes accurately, quickly and in real time not only can optimize the production and construction of materials, but also provide strong support for its engineering application.

### BRIEF SUMMARY OF THE INVENTION

The purpose of the one or more embodiments of the invention is to provide a method for monitoring fibers inside concretes, a system, a medium and a product, which can monitor fibers inside concretes accurately, rapidly and quantitatively.

In order to achieve the above purpose, the at least one embodiment provides the following technical scheme.

A method for monitoring fibers inside concretes is provided, where the method for monitoring fibers inside concretes includes:
acquiring a concrete to be measured;
measuring a capacitance value of the concrete to be measured by using a capacitive sensor;
inputting the capacitance value of the concrete to be measured into a permittivity calculation formula to obtain a permittivity of the concrete to be measured;
acquiring a permittivity of a target concrete and a permittivity of a fiber-free concrete; and
inputting the permittivity of the concrete to be measured, the permittivity of the target concrete and the permittivity of the fiber-free concrete into a prediction model, so as to obtain a measured fiber direction effective coefficient of the concrete to be measured as a monitoring result of the concrete to be measured.

In some embodiments, the permittivity calculation formula is:

$${ε}_{measured} = Cd / S ;$$

where d represents a distance between two electrodes of the capacitive sensor; S represents the area of two electrodes of the capacitive sensor, and C represents the capacitance value of the concrete to be measured; ε_{measured} represents the permittivity of the concrete to be measured. In some embodiments, the prediction model is:

$$Δ ε = {ε}_{m easured} - {ε}_{1} ;$$

$${K}_{1} = Δε / ( {ε}_{2} - {ε}_{1} ) ;$$

where ε_{measured} represents the permittivity of the concrete to be measured; Δε represents an increase of a relative permittivity resulted from fibers; ε₁ represents the permittivity of the fiber-free concrete; ε₂ represents the permittivity of the target concrete; K₁ represents the measured fiber direction effective coefficient of the concrete to be measured.

In some embodiments, the method for monitoring fibers inside concretes further includes:
scanning the concrete to be measured to obtain image data of the concrete to be measured;
obtaining fiber data of the concrete to be measured based on the image data; where the fiber data includes the number of fibers, the length of fibers and an included angle between each fiber and a normal line of a cross section;
inputting the fiber data of the concrete to be measured into a first fiber effective coefficient relational model to obtain a scanned fiber direction effective coefficient of the concrete to be measured.

In some embodiments, the first fiber effective coefficient relational model is:

$${K}_{X - C T} = \frac{{\sum }_{i = 1}^{n} l \times cos {θ}_{i}}{n \times l} = \frac{1}{n} {\sum }_{i = 1}^{n} cos ( {θ}_{i} ) ;$$

where K_{X-CT} represents the scanned fiber direction effective coefficient of the concrete to be measured; n represents the number of fibers; l represents a length of an i-th fiber; cos θᵢ represents the included angle between the i-th fiber and the normal line of the cross section.

In some embodiments, the method for monitoring fibers inside concretes further includes:
inputting the measured fiber direction effective coefficient of the concrete to be measured into a second fiber effective coefficient relational model to obtain the scanned fiber direction effective coefficient of the concrete to be measured.

In some embodiments, the second fiber effective coefficient relational model is:

$${K}_{1} = a \times {K}_{X - CT} + b ;$$

where K₁ represents a fiber direction effective coefficient of the concrete to be measured; a represents a first fitting parameter; b represents a second fitting parameter; K_{X-CT} indicates the scanned fiber direction effective coefficient of the concrete to be measured.

A computer system is provided, including a memory, a processor, and a computer program which is stored on the memory and executable on the processor, where the processor executes the computer program to implement steps of the method for monitoring fibers inside concretes described above.

A non-transient computer-readable storage medium is provided, on which a computer program is stored, where the computer program, when executed by a processor, implements the steps of the method for monitoring fibers inside concretes described above.

A computer program product is provided, including a computer program, where the computer program, when executed by a processor, implements the steps of the method for monitoring fibers inside concretes described above.

Compared with the prior art, one or more embodiments of the invention has the following beneficial effects.

At least one embodiment discloses a method for monitoring fibers inside concretes, a system, a medium and a product. The method includes: acquiring a concrete to be measured; measuring a capacitance value of the concrete to be measured by using a capacitive sensor; inputting the capacitance value of the concrete to be measured into a permittivity calculation formula to obtain a permittivity of the concrete to be measured; acquiring a permittivity of a target concrete and a permittivity of a fiber-free concrete; and inputting the permittivity of the concrete to be measured, the permittivity of the target concrete and the permittivity of the fiber-free concrete into a prediction model, so as to obtain a measuring fiber direction effective coefficient of the concrete to be measured as a monitoring result of the concrete to be measured. The present invention can monitor fibers inside concretes accurately, rapidly and quantitatively.

### BRIEF DESCRIPTION OF THE DRAWINGS

The one or more embodiments will be further described with reference to the accompanying drawings.

FIG. 1 is a flow schematic diagram of a method for monitoring fibers inside concretes according to one or more embodiments of the invention.

FIG. 2 is a schematic diagram of fiber preparation and arrangement according to one or more embodiments of the invention.

FIG. 3 is a schematic diagram of a detection principle of a capacitance value according to one or more embodiments of the invention.

FIG. 4 is a flow schematic diagram of an operation process according to one or more embodiments of the invention.

### DETAILED DESCRIPTION OF THE INVENTION

The technical solutions in the embodiments of the invention will be described in detail with reference to the drawings in the embodiments of the present disclosure hereinafter. Obviously, the described embodiments are only some embodiments of the invention, rather than all of the embodiments. Based on the embodiment of the present disclosure, all other embodiments obtained by those skilled in the art without creative labor fall within the scope of protection of the one or more embodiments of the invention.

The purpose of the one or more embodiments of the invention is to provide a method for monitoring fibers inside concretes, a system, a medium and a product, aiming at realizing visual quantitative detection of the fiber distribution within fiber concretes and providing theoretical support for performance optimization and long-term stability of fiber concretes.

The capacitive sensor involved in the one or more embodiments of the invention is a variable permittivity-type capacitive sensor, and its detection principle is as follows. Concretes and fibers have different permittivity. When fibers are mixed into concretes, the permittivity of the mixed medium (fiber concretes) will change, thereby causing the change of capacitance value. The information about the number of fibers inside concretes can be obtained by acquiring the signal change through the capacitive sensor. At the same time, the change of the fiber direction inside concretes measured by the capacitive sensor will affect the distribution of the electric field and also causes the change of the measured capacitance signal. The information about the fiber direction inside concretes can be obtained by establishing a quantitative equation to analyze the change of the capacitance signal. Since the concrete is an insulator and the steel is a conductor, there is a significant difference in their ability to store charge. Therefore, a capacitance method is more sensitive to the existence and state change of fibers inside the concretes, and can effectively provide researchers with quantitative information about the distribution of fibers inside concretes.

In order to make the above objects, features and advantages of the one or more embodiments of the invention more apparent, the one or more embodiments will be further described in detail in conjunction with the attached drawings and the specific implementation.

### Embodiment 1

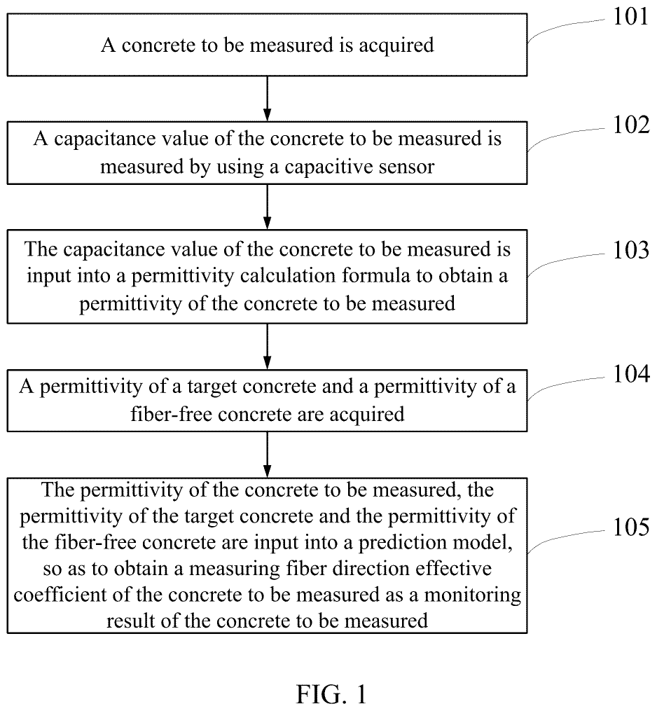

As shown in FIG. 1, in this embodiment, a method for monitoring fibers inside concretes is provided. The method for monitoring fibers inside concretes includes the following steps 101-105.

In step 101, a concrete to be measured is acquired.

In step 102, a capacitance value of the concrete to be measured is measured by using a capacitive sensor.

In step 103, the capacitance value of the concrete to be measured is input into a permittivity calculation formula to obtain a permittivity of the concrete to be measured.

The formula of calculating the permittivity is:

$${ε}_{measured} = Cd / S ;$$

where d represents a distance between two electrodes of the capacitive sensor; S represents the area of two electrodes of the capacitive sensor, and C represents the capacitance value of the concrete to be measured; ε_{measured} represents the permittivity of the concrete to be measured.

In step 104, a permittivity of a target concrete and a permittivity of a fiber-free concrete are acquired.

The target concrete is a concrete in an ideal state. The preparation method is to add some concretes to the bottom of a mold, and then lay horizontally arranged fibers on this basis to test the influence of the horizontally arranged fibers on the capacitance value in a relatively ideal state. After pouring is completed, concretes are fully compacted by jolting to ensure that there are no bubbles in the concrete slurry. After solidifying for 24 hours, a member with a predetermined shape is obtained by being subjected to gently demoulding. In order to prepare for further experiments, a concrete test block is placed in a standard curing room for 28 days. After being cleaned, these blocks are dried until they have a constant weight, so as to prepare for the follow-up experiments.

In step 105, the permittivity of the concrete to be measured, the permittivity of the target concrete and the permittivity of the fiber-free concrete are input into a prediction model, so as to obtain a measured fiber direction effective coefficient of the concrete to be measured as a monitoring result of the concrete to be measured.

The prediction model is:

$$Δε = {ε}_{measured} - {ε}_{1} ;$$

$${K}_{1} = Δε / ( {ε}_{2} - {ε}_{1} ) ;$$

where ε_{measured} represents the permittivity of the concrete to be measured; Δε represents an increase of a relative permittivity resulted from fibers; ε₁ represents the permittivity of the fiber-free concrete; ε₂ represents the permittivity of the target concrete; K₁ represents the measured fiber direction effective coefficient of the concrete to be measured.

K₁ reflects the effectiveness of fiber arrangement inside concretes, and a higher K₁ value indicates that the fiber arrangement is close to the ideal state, which may effectively improve the performance of concretes. On the contrary, a lower K₁ value may mean that the fiber arrangement effect needs to be optimized. Statistical analysis is performed on the K₁ values calculated from all test blocks to quantitatively analyze the fiber arrangement inside concretes, so as to optimize the mechanical performance and the durability of concretes.

As an embodiment, the method for monitoring fibers inside concretes further includes:
scanning the concrete to be measured to obtain image data of the concrete to be measured;
obtaining fiber data of the concrete to be measured based on the image data; wherein the fiber data includes the number of fibers, the length of fibers and an included angle between each fiber and a normal of a cross section;
inputting the fiber data of the concrete to be measured into a first fiber effective coefficient relational model to obtain a scanned fiber direction effective coefficient of the concrete to be measured.

The relational model of the first fiber effective coefficient is:

$${K}_{X - CT} = \frac{{\sum }_{i = 1}^{n} l \times cos {θ}_{i}}{n \times l} = \frac{1}{n} {\sum }_{i = 1}^{n} cos ( {θ}_{i} ) ;$$

where K_{X-CT} represents the scanned fiber direction effective coefficient of the concrete to be measured; n represents the number of fibers; l represents the length of fibers; cos θᵢ represents the included angle between an i-th fiber and the normal line of the cross section.

As an embodiment, the method for monitoring fibers inside concretes further includes the following steps:
inputting the measuring fiber direction effective coefficient of the concrete to be measured into a second fiber effective coefficient relational model to obtain the scanned fiber direction effective coefficient of the concrete to be measured.

The second fiber effective coefficient relational model is:

$${K}_{1} = a \times {K}_{X - CT} + b ;$$

where K₁ represents a fiber direction effective coefficient of the concrete to be measured; a represents a first fitting parameter; b represents a second fitting parameter; K_{X-CT} indicates a scanned fiber direction effective coefficient of the concrete to be measured.

The present disclosure provides an embodiment to prove that the present disclosure can monitor fibers inside concretes accurately, rapidly and quantitatively. Taking the monitoring of steel fiber reinforced concrete (SFRC) as an example, but not limited to steel fiber reinforced concretes, the method can also be used to monitor other similar building materials (such as corrosion of concrete reinforcement, medium transmission, etc.) that can result in changes in the capacitance value of test blocks.

### (1) Preparing a Concrete Test Block

In order to avoid the adverse effect of vibration on the arrangement of steel fibers, self-compacting concretes are prepared by mixing a proper amount of cement, fine aggregate and water according to the standardized ratio with a certain water cement ratio and steel fiber volume content. The mold that meets the size requirements is selected, cleaned and greased to facilitate the demoulding of the test block. Two pouring methods are used. The first method is to add part of concretes to the bottom of the mold, and then lay horizontally arranged steel fibers on this basis to test the influence of horizontally arranged steel fibers on the capacitance value in a relatively ideal state. The second method is to use an L-shaped pouring device to induce the steel fiber orientation by flow, so as to test the change of the capacitance value of steel fiber oriented concretes in practical engineering.

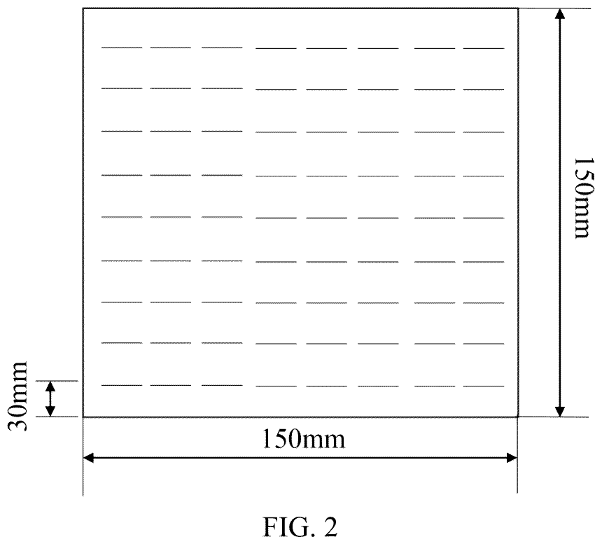

The first method is used to prepare the target steel fiber reinforced concrete test block, that is, the steel fiber reinforced concrete test block in the ideal state, as shown in FIG. 2. The second method is used to prepare sample steel fiber reinforced concrete test blocks, which can prepare a plurality of sample steel fiber reinforced concrete test blocks.

Before the initial setting of concretes, each test block is numbered, and the arrangement angle and the laying sequence of steel fibers of each test block are recorded. After concrete pouring is completed, the test block is covered with plastic wrap or wet linen, and placed in a curing box that maintains a certain temperature and humidity condition, so that the test block solidifies within 24 hours. After solidification, the test block is demoulded gently, and placed in the standard curing room for further curing for 28 days, and then dried to a constant weight to reduce the test error resulted from moisture.

### (2) Measuring a Permittivity

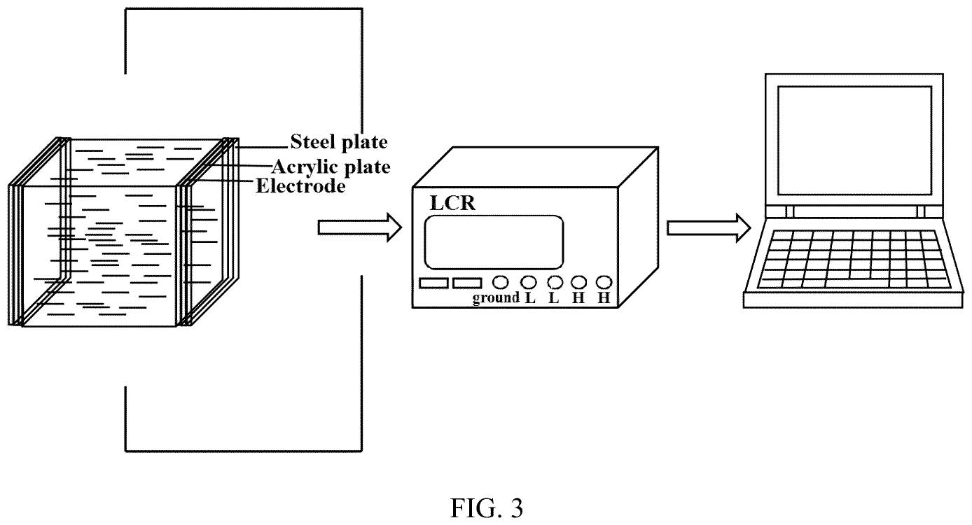

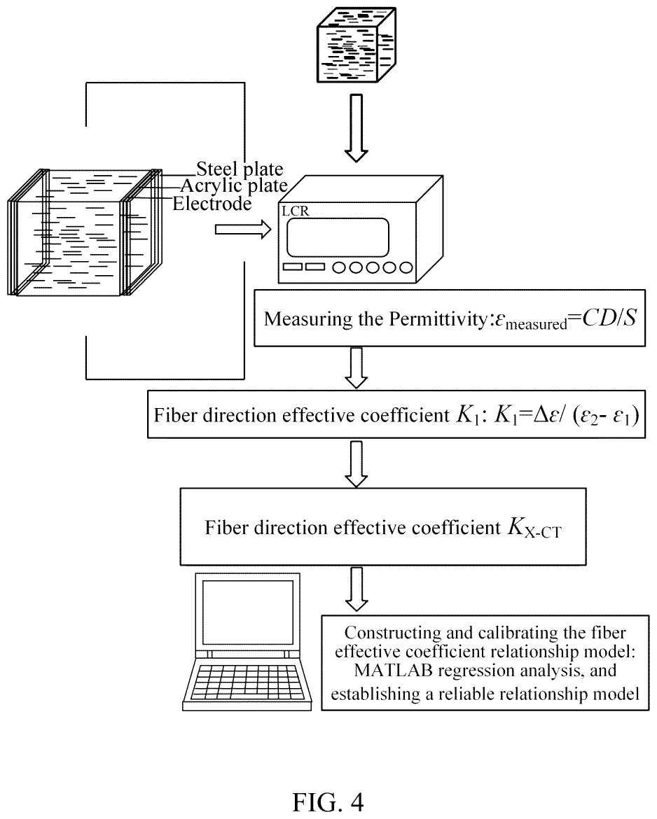

As shown in FIG. 3 and FIG. 4, the test environment is first set to ensure that each test block is measured under the same temperature and humidity conditions. Prior to the test, the capacitance measuring device (an LCR digital bridge can be used) is preheated for about 30 minutes to stabilize its performance. Each test block is numbered prior to measurement, which is convenient for subsequent data tracking and analysis. Thereafter, the parallel plate capacitive sensor is connected to the digital bridge through electrode leads, ensuring that one electrode is connected to the L port, the other electrode is connected to the H port, and the shielding device is connected to the ground port.

The test block is taken out from the standard curing room. In order to calculate the increase of the permittivity, it is necessary to measure the permittivity value of a steel fiber-free concrete test block as a benchmark. In this way, by comparing the permittivity values of the steel fiber reinforced concrete test blocks and the steel fiber reinforced concrete test blocks in the ideal state, the change of the permittivity resulted from steel fiber arrangement can be determined. The capacitance value of the fiber-free concrete test block is measured as the reference value ε₁ of the permittivity. Thereafter, the steel fiber reinforced concrete test block in the ideal state and the sample steel fiber reinforced concrete test block are placed in the capacitive sensor in sequence and are fixed by the fixture to ensure the consistency and the accuracy of the measurement. The digital bridge is turned on, and the capacitance value of the steel fiber-free concrete test block is recorded as a benchmark. Subsequently, the capacitance values of the steel fiber reinforced concrete test blocks in the ideal state and the sample steel fiber reinforced concrete test blocks are measured and recorded carefully. Each test block should be measured at least three times to calculate the average capacitance value and reduce the influence of random errors. According to the distance d between two electrodes of the capacitive sensor and the area S of the electrode and the measured capacitance value C, the permittivity ε of each test block is obtained by conversion using the Formula ε=Cd/S. The specific influence of fiber arrangement on the dielectric property is determined by comparing the permittivity values of various test block. By comparing the permittivity values of the steel fiber reinforced concrete test blocks in the ideal state and the sample steel fiber reinforced concrete test blocks, the change of the permittivity resulted from steel fiber arrangement can be determined. The capacitance value of the steel fiber-free concrete test block is measured as the reference value ε₁ of the permittivity, and the capacitance value ε₂ of the steel fiber reinforced concrete test blocks in the ideal state is measured, and the capacitance value ε_{measured} of the sample steel fiber reinforced concrete test block is measured.

### (3) Calculating the Steel Fiber Direction Effective Coefficient

In order to calculate the fiber direction effective coefficient in detail, first, ε₂ is obtained by the method of (2), and the influence of steel fibers on dielectric properties is calculated accordingly. Based on Δε of each test block obtained in (2), the fiber direction effective coefficient K₁ is calculated thereafter, and is defined as the ratio of the change of the relative permittivity to the capacitance value of steel fibers in the ideal horizontal arrangement state: K₁=Δε/(ε₂−ε₁).

Based on the obtained permittivity, K₁ is calculated. Based on the Δε of each test block obtained in (2), K₁ is calculated thereafter, where K₁ is defined as the ratio of the change of the relative permittivity to the capacitance value of fibers in the ideal horizontal arrangement, that is, K₁=Δε/(ε₂−ε₁). Through the statistical analysis of K₁ values calculated from all test blocks, the fiber arrangement inside concretes can be quantitatively analyzed, and furthermore, the influence of different arrangement states on the concrete performance can be further understood through the correlation analysis with concrete mechanics and durability indexes.

### (4) Building and Calibrating the Fiber Effective Coefficient Relational Model

X-CT scanning is carried out on the sample steel fiber reinforced concrete test blocks after capacitance measurement, so as to provide high-resolution three-dimensional images of the inner part of the sample steel fiber reinforced concrete test blocks and reveal the spatial distribution and direction of fibers. Through X-Ray Computed Tomography (X-CT) image data, the spatial position and direction of each fiber in the test blocks are analyzed and counted. The statistical data includes the number of fibers and the included angle θᵢ between each fiber and the normal line of the cross section, and K_{X-CT} is calculated according to the Formula

$${K}_{X - CT} = \frac{{\sum }_{i = 1}^{n} l \times cos {θ}_{i}}{n \times l} = \frac{1}{n} {\sum }_{i = 1}^{n} cos ( {θ}_{i} ) .$$

The K₁ and K_{X-CT} of each test block are compared and analyzed, and the data of K₁ and K_{X-CT} are regressed and analyzed by using the MATLAB software. A reliable relational model K₁=a×K_{X-CT}+b (where a and b are regression parameters) is established. The reliable relational model includes but is not limited to the form of a linear function. By calculating and comparing the mean square errors of each model, the difference between the predicted value and the actual observed value of the model is quantified. The smaller the error, the higher the accuracy and reliability of the model. By comparing the mean square errors of different models, the model with the smallest error can be selected as the optimal relational model for evaluating the arrangement of steel fibers inside concretes. The established reliable relational model is used to calibrate and verify the fiber direction effective coefficient obtained by the capacitance method, so as to improve the accuracy of arrangement evaluation of steel fibers inside concretes.

Compared with the prior art, the one or more embodiments of the invention has the following advantages.
(1) The one or more embodiments of the invention uses the capacitance measurement technology to monitor fiber arrangement, which will not result in any invasive damage to concretes and will not affect the performance and the service life of the material itself.
(2) The one or more embodiments of the invention can monitor the arrangement of fibers inside concretes in real time through the change of the electromagnetic field, provide continuous data, monitor the dynamic change of arrangement of fibers inside concretes with time in real time, and provide a more convenient method for studying the influence and evaluation of an external environment such as different environments and loads on arrangement of steel fibers inside concretes.
(3) The one or more embodiments of the invention calculates K₁ based on the capacitance measurement technology, which provides an innovative technical approach for accurately evaluating the arrangement of fibers inside steel fiber reinforced concretes. By quantifying the influence of steel fibers on the dielectric properties of concretes, the effectiveness of fiber arrangement can be intuitively judged, so as to quickly evaluate the structural properties of concretes and improve the testing efficiency without destroying materials.
(4) By combining the capacitance measurement technology and the X-ray computer tomography, the one or more embodiments of the invention can evaluate arrangement of steel fibers inside concretes more comprehensively and accurately. This data fusion not only enhances the accuracy of fiber arrangement evaluation, but also establishes the relational model between the two technologies through regression analysis with the MATLAB software to effectively calibrate data. This innovative method not only improves the evaluation efficiency, but also provides a more scientific basis for the design and construction of concrete structures.

To sum up, the one or more embodiments of the invention uses the advanced capacitance measurement technology to monitor and analyze the arrangement of fibers inside concretes. Compared with traditional monitoring methods such as a resistance measurement method, the capacitance measurement technology can provide data of fiber distribution in real time without damaging concrete structures, which greatly enriches the depth and the breadth of material testing and evaluation. Based on the change of the permittivity, a formula of calculating the fiber direction effective coefficient with the permittivity as a variable is proposed. Combined with the X-CT technology, the fiber direction effective coefficient is obtained by comparing the two methods and the relational model between the parameters obtained by the two technologies is established, thus providing a comprehensive and accurate perspective for the evaluation of the internal structure of concretes.

### Embodiment 2

A computer system is provided, including a memory, a processor, and a computer program which is stored on the memory and executable on the processor, where the processor executes the computer program to implement the steps of the method for monitoring fibers inside concretes in Embodiment 1.

### Embodiment 3

A non-transient computer-readable storage medium is provided, on which a computer program is stored, where the computer program, when executed by a processor, implements the steps of the method for monitoring fibers inside concretes in Embodiment 1.

### Embodiment 4

A computer program product is provided, including a computer program, where the computer program, when executed by a processor, implements the steps of the method for monitoring fibers inside concretes in Embodiment 1.

### Embodiment 5

A computer device is provided, which can be a database. The computer device includes a processor, a memory, an Input/Output interface (I/O for short) and a communication interface. The processor, the memory and the input/output interface are connected through a system bus, and the communication interface is connected to the system bus through the input/output interface. The processor of the computer device is configured to provide computing and control capabilities. The memory of the computer device includes a non-volatile storage medium and an internal memory. The non-volatile storage medium stores an operating system, a computer program and a database. The internal memory provides an environment for the operation of the operating system and the computer program in the non-volatile storage medium. The database of the computer device is configured to store pending transactions. The input/output interface of the computer device is configured to exchange information between the processor and the external device. The communication interface of the computer device is configured to communicate with an external terminal through network connection. The computer program, when executed by a processor, implements the method for monitoring fibers inside concretes in Embodiment 1.

It should be noted that the object information (including but not limited to object device information, object personal information, etc.) and data (including but not limited to analyzed data, stored data, displayed data, etc.) involved in one or more embodiments of the invention are all information and data authorized by the object or fully authorized by all parties, and the collection, use and processing of relevant data need to comply with relevant laws, regulations and standards of relevant countries and regions.

Those skilled in the art can understand that all or part of the processes in the method of implementing the above-mentioned embodiments can be completed by instructing related hardware through a computer program. The computer program can be stored in a non-volatile computer-readable storage medium. When executed, the computer program can include the processes of the embodiments of each method described above. Any reference to the memory, the database or other media used in various embodiments provided by the one or more embodiments of the invention may include at least one of a non-volatile memory and a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto-Resistive Random Access Memory (MRAM), a Ferroelectric Random Access Memory (FRAM), a Phase Change Memory (PCM), a graphene memory, etc. The volatile memory may include a Random Access Memory (RAM) or an external cache memory. By way of illustration and not limitation, RAM can be in various forms, such as a Static Random Access Memory (SRAM) or a Dynamic Random Access Memory (DRAM). The database involved in each embodiment provided by the one or more embodiments of the invention may include at least one of a relational database and a non-relational database. Non-relational databases may include, but are not limited to, distributed databases based on blockchain. The processors involved in various embodiments provided by the one or more embodiments of the invention may be general processors, central processing units, graphics processors, digital signal processors, programmable logics, data processing logics based on quantum computing, etc., but are not limited thereto.

The technical features of the above embodiments can be combined at will. In order to make the description concise, not all possible combinations of each technical feature in the above embodiments are described. However, as long as there is no contradiction between the combinations of these technical features, these technical features should be considered as the scope described in this specification.

In the one or more embodiments of the invention, specific examples are applied to illustrate the principle and implementation of the one or more embodiments, and the explanations of the above embodiments are only used to help understand the method and core ideas of the invention. At the same time, according to the idea of the one or more embodiments of the invention, there will be some changes in the specific implementation and the application scope for those skilled in the art. To sum up, the contents of the specification should not be construed as limiting the one or more embodiments of the invention.

1-10. (canceled)

11. A method for monitoring fibers inside concretes, comprising:
acquiring a concrete to be measured;
measuring a capacitance value of the concrete to be measured by using a capacitive sensor;
inputting the capacitance value of the concrete to be measured into a permittivity calculation formula to obtain a permittivity of the concrete to be measured;
acquiring a permittivity of a target concrete and a permittivity of a fiber-free concrete; and
inputting the permittivity of the concrete to be measured, the permittivity of the target concrete and the permittivity of the fiber-free concrete into a prediction model, so as to obtain a measured fiber direction effective coefficient of the concrete to be measured as a monitoring result of the concrete to be measured.

12. The method for monitoring fibers inside concretes according to claim 11, wherein the permittivity calculation formula is:
$${ε}_{measured} = Cd / S ;$$
where d represents a distance between two electrodes of the capacitive sensor; S represents an area of the two electrodes of the capacitive sensor, and C represents the capacitance value of the concrete to be measured; ε_{measured} represents the permittivity of the concrete to be measured.

13. The method for monitoring fibers inside concretes according to claim 11, wherein the prediction model is:
$$Δε = {ε}_{measured} - {ε}_{1} ;$$
$${K}_{1} = Δε / ( {ε}_{2} - {ε}_{1} ) ;$$
where ε_{measured} represents the permittivity of the concrete to be measured; Δε represents an increase of a relative permittivity resulted from fibers; ε₁ represents the permittivity of the fiber-free concrete; ε₂ represents the permittivity of the target concrete; K₁ represents the measured fiber direction effective coefficient of the concrete to be measured.

14. The method for monitoring fibers inside concretes according to claim 11, further comprising:
scanning the concrete to be measured to obtain image data of the concrete to be measured;
obtaining fiber data of the concrete to be measured based on the image data; wherein the fiber data comprises a number of fibers, a length of each fiber and an included angle between each fiber and a normal line of a cross section;
inputting the fiber data of the concrete to be measured into a first fiber effective coefficient relational model to obtain a scanned fiber direction effective coefficient of the concrete to be measured.

15. The method for monitoring fibers inside concretes according to claim 14, wherein the first fiber effective coefficient relational model is:
$${K}_{X - CT} = \frac{{\sum }_{i = 1}^{n} l \times cos {θ}_{i}}{n \times l} = \frac{1}{n} {\sum }_{i = 1}^{n} cos ( {θ}_{i} ) ;$$
where K_{X-CT} represents the scanned fiber direction effective coefficient of the concrete to be measured; n represents the number of fibers; l represents a length of an i-th fiber; cos θᵢ represents the included angle between the i-th fiber and the normal line of the cross section.

16. The method for monitoring fibers inside concretes according to claim 11, further comprising:
inputting the measured fiber direction effective coefficient of the concrete to be measured into a second fiber effective coefficient relational model to obtain a scanned fiber direction effective coefficient of the concrete to be measured.

17. The method for monitoring fibers inside concretes according to claim 16, wherein the second fiber effective coefficient relational model is:
$${K}_{1} = a \times {K}_{X - CT} + b ;$$
where K₁ represents a fiber direction effective coefficient of the concrete to be measured; a represents a first fitting parameter; b represents a second fitting parameter; K_{X-CT} indicates the scanned fiber direction effective coefficient of the concrete to be measured.

18. A computer system, comprising a memory, a processor, and a computer program which is stored on the memory and executable on the processor, wherein the processor executes the computer program to implement steps of the method for monitoring fibers inside concretes according to any one of claim 11.

19. A non-transient computer-readable storage medium on which a computer program is stored, wherein the computer program, when executed by a processor, implements steps of the method for monitoring fibers inside concretes according to any one of claim 11.

20. The computer system according to claim 18, wherein the permittivity calculation formula is:
$${ε}_{measured} = Cd / S ;$$
where d represents a distance between two electrodes of the capacitive sensor; S represents an area of the two electrodes of the capacitive sensor, and C represents the capacitance value of the concrete to be measured; ε_{measured} represents the permittivity of the concrete to be measured.

21. The computer system according to claim 18, wherein the prediction model is:
$$Δε = {ε}_{measured} - {ε}_{1} ;$$
$${K}_{1} = Δε / ( {ε}_{2} - {ε}_{1} ) ;$$
where ε_{measured} represents the permittivity of the concrete to be measured; Δε represents an increase of a relative permittivity resulted from fibers; ε₁ represents the permittivity of the fiber-free concrete; ε₂ represents the permittivity of the target concrete; K₁ represents the measured fiber direction effective coefficient of the concrete to be measured.

22. The computer system according to claim 18, further comprising:
scanning the concrete to be measured to obtain image data of the concrete to be measured;
obtaining fiber data of the concrete to be measured based on the image data; wherein the fiber data comprises a number of fibers, a length of each fiber and an included angle between each fiber and a normal line of a cross section;
inputting the fiber data of the concrete to be measured into a first fiber effective coefficient relational model to obtain a scanned fiber direction effective coefficient of the concrete to be measured.

23. The computer system according to claim 22, wherein the first fiber effective coefficient relational model is:
$${K}_{X - CT} = \frac{{\sum }_{i = 1}^{n} l \times cos {θ}_{i}}{n \times l} = \frac{1}{n} {\sum }_{i = 1}^{n} cos ( {θ}_{i} ) ;$$
where K_{X-CT} represents the scanned fiber direction effective coefficient of the concrete to be measured; n represents the number of fibers; l represents a length of an i-th fiber; cos θᵢ represents the included angle between the i-th fiber and the normal line of the cross section.

24. The computer system according to claim 18, further comprising:
inputting the measured fiber direction effective coefficient of the concrete to be measured into a second fiber effective coefficient relational model to obtain a scanned fiber direction effective coefficient of the concrete to be measured.

25. The computer system according to claim 24, wherein the second fiber effective coefficient relational model is:
$${K}_{1} = a \times {K}_{X - CT} + b ;$$
where K₁ represents a fiber direction effective coefficient of the concrete to be measured; a represents a first fitting parameter; b represents a second fitting parameter; K_{X-CT} indicates the scanned fiber direction effective coefficient of the concrete to be measured.

26. The non-transient computer-readable storage medium according to claim 19, wherein the permittivity calculation formula is:
$${ε}_{measured} = Cd / S ;$$
where d represents a distance between two electrodes of the capacitive sensor; S represents an area of the two electrodes of the capacitive sensor, and C represents the capacitance value of the concrete to be measured; ε_{measured} represents the permittivity of the concrete to be measured.

27. The non-transient computer-readable storage medium according to claim 19, wherein the prediction model is:
$$Δε = {ε}_{measured} - {ε}_{1} ;$$
$${K}_{1} = Δε / ( {ε}_{2} - {ε}_{1} ) ;$$
where ε_{measured} represents the permittivity of the concrete to be measured; Δε represents an increase of a relative permittivity resulted from fibers; ε₁ represents the permittivity of the fiber-free concrete; ε₂ represents the permittivity of the target concrete; K₁ represents the measured fiber direction effective coefficient of the concrete to be measured.

28. The non-transient computer-readable storage medium according to claim 19, further comprising:
scanning the concrete to be measured to obtain image data of the concrete to be measured;
obtaining fiber data of the concrete to be measured based on the image data; wherein the fiber data comprises a number of fibers, a length of each fiber and an included angle between each fiber and a normal line of a cross section;
inputting the fiber data of the concrete to be measured into a first fiber effective coefficient relational model to obtain a scanned fiber direction effective coefficient of the concrete to be measured.

29. The non-transient computer-readable storage medium according to claim 19, wherein the first fiber effective coefficient relational model is:
$${K}_{X - CT} = \frac{{\sum }_{i = 1}^{n} l \times cos {θ}_{i}}{n \times l} = \frac{1}{n} {\sum }_{i = 1}^{n} cos ( {θ}_{i} ) ;$$
where K_{X-CT} represents the scanned fiber direction effective coefficient of the concrete to be measured; n represents the number of fibers; l represents a length of an i-th fiber; cos θᵢ represents the included angle between the i-th fiber and the normal line of the cross section.

30. The non-transient computer-readable storage medium according to claim 19, further comprising:
inputting the measured fiber direction effective coefficient of the concrete to be measured into a second fiber effective coefficient relational model to obtain a scanned fiber direction effective coefficient of the concrete to be measured.
